(19) United States
(12) Patent Application Publication
(10) Pub. No.: US 2010/0227811 A1
(43) Pub. Date: Sep. 9, 2010

(54) FARNESYLAMINE DERIVATIVES AND METHODS OF USE

(75) Inventors: Ravi Kothapalli, Wesley Chapel, FL (US); Kirpal S. Bisht, Tampa, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of South Florida, Tampa, FL (US)

(21) Appl. No.: 12/599,013

(22) PCT Filed: May 14, 2008
(86) PCT No.: PCT/US2008/006251

§ 371 Date: Apr. 19, 2010

Related U.S. Application Data
(60) Provisional application No. 60/917,861, filed on May 14, 2007.

(51) Int. Cl. A61K 31/16; C07D 209/48; C07C 233/65; C07C 233/02; A61K 31/4015; A61K 31/166; A61K 38/02; A61K 38/18; A61K 31/56; A01N 43/38; A01N 37/18; A61P 35/00; A01P 1/00

(52) U.S. Cl. 514/12; 548/480; 564/156; 564/215; 554/35; 514/417; 514/616; 514/629; 514/2; 514/171

(57) ABSTRACT

The present invention concerns farnesylamine derivatives, compositions containing these compounds, and methods of using these compounds and compositions as inhibitors of ras-mediated signal transduction and inhibitors of aberrant cell growth, e.g., as anti-cancer agents, as well as anti-fungal agents. Other non-malignant diseases characterized by proliferation of cells that may be treated using the farneylamine derivatives of the include, but are not limited to, cirrhosis of the liver; graft rejection; restenosis; and disorders characterized by a proliferation of T cells such as autoimmune diseases, e.g., type 1 diabetes, lupus and multiple sclerosis.

### CROSS-REFERENCE TO RELATED APPLICATION

The present application claims the benefit of U.S. Provisional Application Ser. No. 60/917,861, filed May 14, 2007, which is hereby incorporated by reference herein in its entirety, including any figures, tables, nucleic acid sequences, amino acid sequences, and drawings.

### BACKGROUND OF THE INVENTION

The family of Ras proteins, including H-, N-, and K-ras, play a central role in the regulation of cell growth. These ras proteins frequently undergo mutations and are responsible for unregulated cell proliferation. Such oncogenically mutated forms are found in many human cancers. The type of mutations and the frequency depends on the type of the tumor. The most common mutation found in the various tumors is K-ras mutation. These mutations are observed in 90% of pancreatic cancers, 50% colon, 35% of head and neck cancers, and 30% of lung cancers. Farnesylation of Ras is necessary for its biological function. Any compound capable of blocking the farnesylation of ras is a potential drug for treating human cancers. Recently, two groups of compounds were developed to prevent the function of mutated ras function. Two groups of these compounds are peptidomimetics and farnesyl derivatives.

Recently, it has been well-established that polyisoprenoid compounds such as farnesol and geranylgeraniol are attached post-translationally to a small group of proteins (Maltese 1990; Maltese et al., 1990a; Maltese et al. 1990b; Maltese et al. 1990c). Some of these proteins, such as Ras proteins (including oncogenic), are biologically active only when they are associated with the inner surface of the plasma membrane (Kato et al. 1992). Members of the ras family of proto-oncogenes are mutated in 30% of human neoplasms (Barbacid 1987; Kiaris et al., 1995). These mutated Ras proteins play an important role in the development and progression of human cancers. A number of reports suggest that both membrane association and biological activity of Ras proteins are abolished by blocking farnesylation of these proteins (Gibbs et al. 1994; Sebti et al. 1997; Cox et al. 1997). In fact, several derivatives of farnesyl compounds such as farnesyl acetate and S-farnesylthiosalicylic acid were also reported as inhibitors of Ras function (Meigs et al. 1995; Gana-Weisz et al. 1997; Overhand et al. 1997; Tahir et al. 2000). It has been demonstrated that farnesylamine, a derivative of farnesol, is a potent inhibitor of farnesylation and growth of ras-transformed cells (Kothapalli et al. 1993; Ura et al. 1998). The amount of farnesylamine required to inhibit cell proliferation by 50% (farnesylamine's IC₅₀ value) is approximately 7.5 μl (Kothapalli et al. 1994).

Farnesylamine is metabolically unstable. It would be advantageous to have available farnesylamine derivatives that are inhibitors of ras and are stable in comparison to farnesylamine.

### BRIEF SUMMARY OF THE INVENTION

The present invention concerns farnesylamine derivatives, compositions containing these compounds, and methods of using these compounds and compositions as inhibitors of ras-mediated signal transduction and inhibitors of aberrant cell growth, e.g., as anti-cancer agents.

In one aspect, the invention includes isolated compounds having the structure of formula A or formula B, or pharmaceutically acceptable salts or analogs thereof (each of which are referred to collectively herein as “farnesylamine derivatives”), wherein R is one or more aliphatic or aromatic functional groups or substituents.

Exemplified embodiments of the invention include the following six farnesylamine derivatives: N-farnesylphthalimide (also referred to herein as compound I); N-methyl-N′-farnesylphthalamide (also referred to herein as compound II); N,N′-bisfarnesylphthalamide (also referred to herein as compound III); farnesylamine acetate (also referred to herein as compound IV); farnesylamine propionate (also referred to herein as compound V); and farnesylamine palmitate (also referred to herein as compound VI). The chemical structures of these compounds (I-VI) are shown in FIG. 2. These compounds are potent inhibitors of cell growth, and are stable in comparison to farnesylamine, thereby being retained in the body for longer periods of time.

### BRIEF DESCRIPTION OF THE DRAWINGS

FIG. 1 shows the generic chemical structure of farnesylamine derivatives of the invention (referred to herein as formula A). R=an aliphatic or aromatic group.

FIG. 2 shows chemical structure of six farnesylamine derivatives: N-farnesylphthalimide (compound I), N-methyl-N′-farnesylphthalamide (compound II), N,N′-bisfarnesylphthalamide (compound III), farnesylamine acetate (compound IV), farnesylamine propionate (compound V), and farnesylamine palmitate (compound VI).

FIG. 3 shows a scheme for the synthesis of compound I.

FIG. 4 shows a scheme for the synthesis of compounds II and III.

FIG. 5 shows a scheme for the synthesis of compounds IV, V, and VI, wherein R═CO₂CH₃ for compound IV; —CO₂CH₂CH₃ for compound V; and —CO₂(CH₂)₁₄CH₃ for compound VI.

FIG. 6 shows a scheme for the synthesis of farnesylamine.

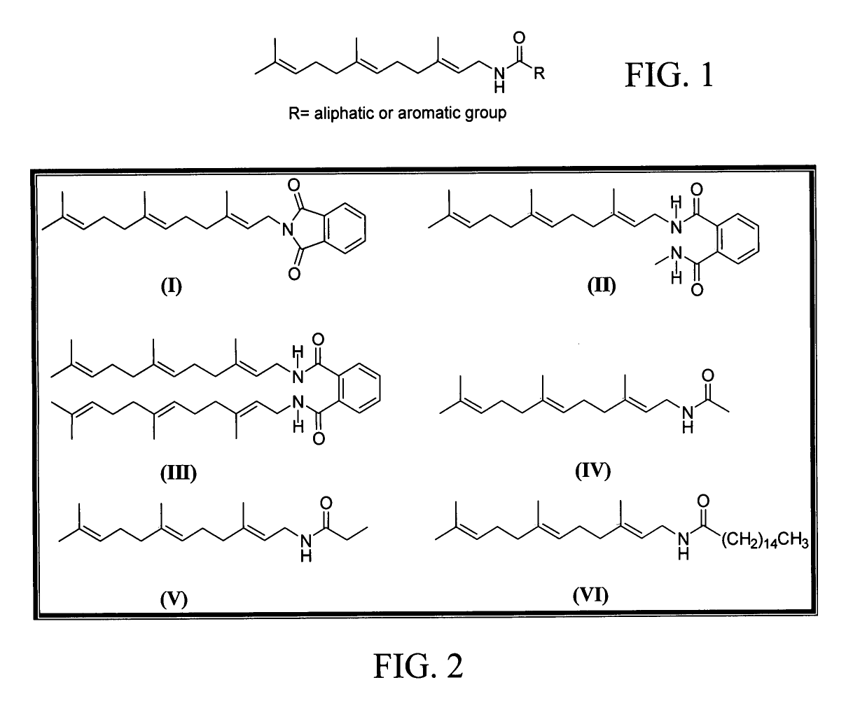

### DETAILED DESCRIPTION OF THE INVENTION

The present invention concerns farnesylamine derivatives, compositions containing these compounds, and methods of using these compounds and compositions as inhibitors of ras-mediated signal transduction and inhibitors of aberrant cell growth, e.g., as anti-cancer agents.

In one aspect, the invention includes isolated compounds having the structure of formula A or B, shown below, or pharmaceutically acceptable salts or analogs thereof (each of which are referred to collectively herein as “farnesylamine derivatives”):

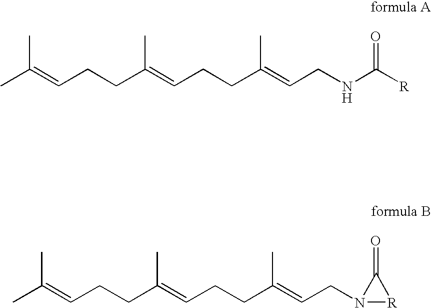

wherein R is one or more aliphatic or aromatic functional groups or substituents optionally substituted with any substituent. In one embodiment, a compound of the invention has the structure of:

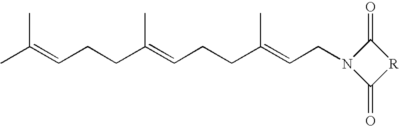

or a pharmaceutically acceptable salt or analog thereof.

In one embodiment, R is one or more of H, alkyl, alkoxy, cycloalkyl, cycloalkoxy, heterocycloalkyl, alkyl-NHC(O)—, acyl, aryl, aryl-NHC(O)—, aryloxy, alkylcarbonyl, alkoxycarbonyl, cycloalkylcarbonyl, cycloalkoxycarbonyl, heteroalkyl, heteroaryl, arylcarbonyl, heteroarylcarbonyl, heterocycloalkylcarbonyl, aryloxycarbonyl, heteroaryloxycarbonyl, heterocycloalkoxy, and/or heterocycloalkoxycarbonyl, any of which can be optionally substituted with one or more of any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

In one embodiment, R is an alkyl group. In a specific embodiment, R is C₁₋₂₀ alkyl. In a further embodiment, R is methyl, ethyl, butyl, pentyl, hexyl, heptyl, octyl, or nonyl.

In another embodiment, R is aryl. In a specific embodiment, R is phenyl.

In specific embodiments, the farnesylamine derivatives of the invention have a structure shown below as N-farnesylphthalimide (compound I), N-methyl-N-farnesylphthalamide (compound II), N,N′-bisfarnesylphthalamide (compound III), farnesylamine acetate (compound IV), farnesylamine propionate (compound V), and farnesylamine palmitate (compound VI); or a pharmaceutically acceptable salt or analog of any of the foregoing:

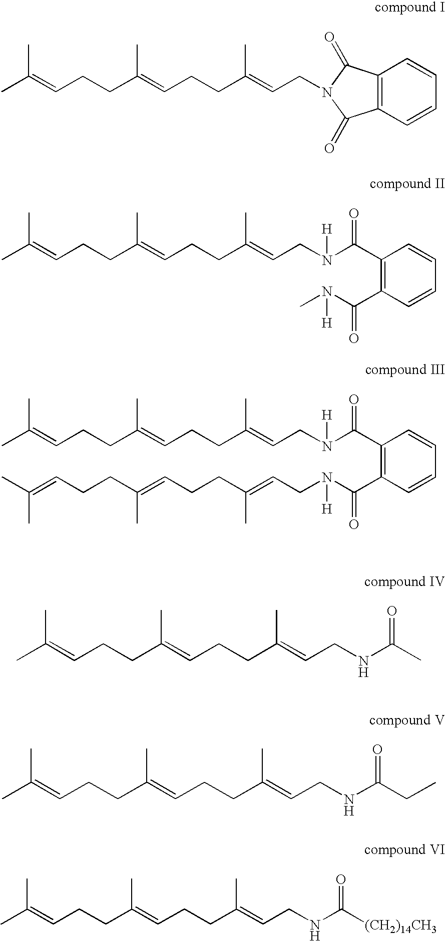

Another aspect of the subject invention concerns methods for using the farnesylamine derivatives of the invention as anti-proliferative agents or anti-fungal agents. Thus, in one embodiment, a method of the invention comprises administering a farnesylamine derivative of the invention, or a composition comprising a farnesylamine derivative, to a human or non-human subject in an amount effective to achieve the desired therapeutic result.

As used herein, the terms “treatment” and “treating”, and grammatical variations thereof, include therapy and prophylaxis. When used as a therapy, the farnesylamine derivatives of the invention, by themselves or in conjunction with other agents, alleviate or reduce one or more symptoms associated with a proliferation disorder (e.g., cancer) or fungal infection. Thus, the treatment methods can be curative or may simply decrease levels of, for example, proliferation or infection. When used as a prophylactic treatment, farnesylamine derivatives of the invention, by themselves or in conjunction with other agents, delay the onset of (and may prevent) one or more symptoms associated with a proliferation disorder (e.g., cancer) or fungal infection.

In one aspect, a method of the invention is directed to treating a cellular proliferation disorder, such as cancer, comprising administering an effective amount of a farnesylamine derivative of the invention, or a composition comprising a farnesylamine derivative, to a subject in need thereof.

In another aspect, a method of the invention is directed to treating a fungal infection, comprising administering an effective amount of a farnesylamine derivative of the invention, or a composition comprising a farnesylamine derivative, to a subject in need thereof.

In another aspect, the invention pertains to antimicrobial compositions comprising one or more farnesylamine derivatives of the invention and the use of farnesylamine derivatives of the invention as an antimicrobial agent. This aspect of the invention concerns methods for disinfecting a substrate (surface), comprising applying an effective amount of one or more farnesylamine derivatives of the invention, or a composition comprising one or more farnesylamine derivatives, to the substrate. Antimicrobial compositions of the invention can include one or more antimicrobial agents in addition to one or more farnesylamine derivatives of the invention.

Another aspect of the present invention concerns methods for inhibiting proliferation of a cell, comprising contacting the cell with an effective amount of one or more farnesylamine derivatives of the invention, or a composition comprising one or more farnesylamine derivatives of the invention. In one embodiment, the cell is a cancerous cell or tumor cell. In a further embodiment, the cell expresses a mutant ras gene. In a specific embodiment, the cell expresses a mutant H-ras, N-ras, and/or K-ras protein. In another embodiment, the cell is a cell of an animal's immune system, e.g., a B cell or T cell. The cell can be a mammalian cell, for example a human cell. In one embodiment, the cell is contacted with one or more of N-farnesylphthalimide (compound I), N-methyl-N′-farnesylphthalamide (compound II), N,N′-bisfarnesylphthalamide (compound III), farnesylamine acetate (compound IV), farnesylamine propionate (compound V), or farnesylamine palmitate (compound VI), and/or pharmaceutically acceptable salts and analogs of the foregoing.

In another aspect, the subject invention provides compositions comprising at least one farnesylamine derivative of the invention, and a pharmaceutically acceptable carrier.

A further aspect of the subject invention provides methods for synthesizing a farnesylamine derivative of the invention.

By inhibiting the growth of cells proliferating in an aberrant or uncontrolled manner, the methods, compounds, and compositions of the present invention can be used to treat cell proliferation disorders, such as cancers, including, but not limited to, leukemias and lymphomas, such as acute lymphocytic leukemia, acute non-lymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms' Tumor, bone tumors, and soft-tissue sarcomas, common solid tumors of adults such as lung cancer, colon and rectum cancer, breast cancer, prostate cancer, urinary cancers, uterine cancers, bladder cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer. The methods of the subject invention can be carried out in vivo or in vitro, to inhibit the growth of cells (e.g., cancer cells) in humans and non-human mammals. Treatment for a proliferation disorder can proceed by the farnesylamine derivative's anti-proliferative activity such as pro-apoptotic activity, or by other mechanisms. In one embodiment, the proliferation disorder is one on which the farnesylamine derivative(s) act by inhibition of farnesylation of proteins, such as ras p21, with the farnesylamine derivative(s) acting as an inhibitor of farnesyl/protein transferase.

In one embodiment, the proliferation disorder to be treated is a cancer characterized as a tumor or cancer cell having a K-ras, H-ras, and/or N-ras mutation. In a specific embodiment, the cancer is characterized as having a K-ras mutation. In a more specific embodiment, the cancer is one having a K-ras mutation and is a type selected from the group consisting of pancreatic, colon, head and neck, and lung.

In addition to cancer, proliferation disorder(s) that can be treated using the compounds, compositions, and methods of the invention include those characterized by Ras-mediated proliferation of cells associated with a non-malignant disease, pathological state or disorder (collectively “disease”). Methods of the invention comprise administering or contacting the cells with an effective amount of one or more farnesylamine derivatives of the invention, or a composition comprising one or more farnesylamine derivatives, to reduce or inhibit the proliferation. The proliferation, hypertrophy or overgrowth of cells that is common to these diseases is mediated by Ras. This protein becomes activated by a series of biochemical events after it binds or docks to a particular site on the inner surface of the cell membrane. The activation of Ras then leads to another series of inter-related biochemical reactions or signal transduction cascades that ultimately produce cell growth and division (U.S. Pat. No. 6,462,086; Kloog et al., 1999).

In one embodiment, the proliferation disorder to be treated is characterized by a proliferation of T-cells such as in an autoimmune disease, e.g., type 1 diabetes, lupus and multiple sclerosis, and pathological states such as graft rejection induced by the presentation of a foreign antigen such as a graft in response to a disease condition (e.g., kidney failure). Other non-malignant diseases contemplated within the scope of the invention and characterized by proliferation of cells include cirrhosis of the liver and restenosis. Treatment of such diseases is contemplated within the scope of the present invention.

The methods of the present invention can be advantageously combined with one or more additional treatment methods, including but not limited to surgery, chemotherapy, radiation therapy, or any other therapy known to those of skill in the art for the treatment and management of proliferation disorders (e.g., cancer) or fungal infections.

In one embodiment, the methods and compositions of the invention include the incorporation of an antagonist of ras or the gene product thereof. Ras protein is the on/off switch between hormone/growth factor receptors and the regulatory cascading that result in cell division. For Ras to be activated (i.e., turned on) to stimulate the regulatory cascades, it must first be attached to the inside of the cell membrane. Ras antagonist drug development aimed at blocking the action of Ras on the regulatory cascades has focused on interrupting the association of Ras with the cell membrane, blocking activation of Ras or inhibiting activated Ras. The details of the approaches to development of Ras antagonists are reviewed in Kloog et al. (1999). Thus, by the term “ras antagonist”, it is meant any compound or agent that targets one or more of these phenomena so as to result in inhibition of cell proliferation.

The Ras antagonists that may be used conjunction with the farnesylamine derivatives of the invention affect (e.g., inhibit) the binding of Ras to the cell membrane, which in turn reduces or inhibits the unwanted cell proliferation. Preferred Ras antagonists include farnesyl thiosalicylic acid (FTS) and structurally related compounds or analogs thereof, which are believed to function by displacing or dislodging Ras from its membrane anchor. These organic compounds may be administered parenterally or orally. In a particularly preferred embodiment, the Ras antagonist is formulated for oral or parenteral administration by complexation with cyclodextrin.

While farnesylamine derivatives of the invention can be administered as isolated compounds, these compounds can also be administered as part of a pharmaceutical composition. The subject invention thus further provides compositions comprising a farnesylamine derivative of the invention, e.g., N-farnesylphthalimide (compound I), N-methyl-N′-farnesylphthalamide (compound II), N,N′-bisfarnesylphthalamide (compound III), farnesylamine acetate (compound IV), farnesylamine propionate (compound V), and/or farnesylamine palmitate (compound VI), or physiologically acceptable salt(s) or analogs of any one or more of the foregoing; in association with at least one pharmaceutically acceptable carrier. In one embodiment, a composition of the invention comprises one or more farnesylamine derivatives of the invention and one or more ras antagonists. The pharmaceutical composition can be adapted for various routes of administration, such as enteral, parenteral, intravenous, intramuscular, topical, subcutaneous, and so forth. Administration can be continuous or at distinct intervals, as can be determined by a person of ordinary skill in the art.

The farnesylamine derivatives of the invention can be formulated according to known methods for preparing pharmaceutically useful compositions. Formulations are described in a number of sources which are well known and readily available to those skilled in the art. For example, Remington's Pharmaceutical Science (Martin 1995) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

In one aspect, the present invention includes isolated compounds having the structure of formula A or B (wherein R in the formula is one or more aliphatic or aromatic groups), or pharmaceutically acceptable salts or analogs thereof (each of which are referred to collectively herein as “farnesylamine derivatives”). For example, R in formula A or B can be one or more of H, alkyl, alkoxy, cycloalkyl, cycloalkoxy, heterocycloalkyl, alkyl NHC(O)—, acyl, aryl, aryl NHC(O)—, aryloxy, alkylcarbonyl, alkoxycarbonyl, cycloalkylcarbonyl, cycloalkoxycarbonyl, heteroalkyl, heteroaryl, arylcarbonyl, heteroarylcarbonyl, heterocycloalkylcarbonyl, aryloxycarbonyl, heteroaryloxycarbonyl, heterocycloalkoxy, and/or heterocycloalkoxycarbonyl, any of which may be optionally substituted with one or more of any halogen, —COOH, —OH, —NO₂, —NH₂, —N-alkyl, alkyl, alkyl NHC(O)—, alkoxy, cycloalkyl, cycloalkoxy, aryl, aryl NHC(O)—, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

As used herein, alkyl means straight or branched chain, saturated or mono- or polyunsaturated hydrocarbon groups having from 1 to 20 carbon atoms and C_{1-X} alkyl means straight or branched chain alkyl groups containing from one up to “X” number of carbon atoms. For example, C₁₋₆ alkyl means straight or branched chain alkyl groups containing from one up to 6 carbon atoms. Alkoxy means an alkyl-O— group in which the alkyl group is as previously described herein. Cycloalkyl includes a nonaromatic monocyclic or multicyclic ring system, including fused and Spiro rings, including rings which may optionally be benzofused at any available position, of from about three to about 10 carbon atoms. A cyclic alkyl may optionally be partially unsaturated. Cycloalkoxy means a cycloalkyl-O— group in which cycloalkyl is as defined herein. Aryl means an aromatic monocyclic or multicyclic carbocyclic ring system, including fused and Spiro rings, containing from about six to about 14 carbon atoms. Aryloxy means an aryl-O— group in which the aryl group is as described herein. Alkylcarbonyl means a RC(O)— group where R is an alkyl group as previously described herein. Alkoxycarbonyl means an ROC(O)— group where R is an alkyl group as previously described herein. Cycloalkylcarbonyl means an RC(O)— group where R is a cycloalkyl group as previously described herein. Cycloalkoxycarbonyl means an ROC(O)— group where R is a cycloalkyl group as previously described herein.

Heteroalkyl means a straight or branched-chain having from one to 20 carbon atoms and one or more heteroatoms selected from nitrogen, oxygen, or sulphur, wherein the nitrogen and sulphur atoms may optionally be oxidized, i.e., in the form of an N-oxide or an S-oxide. Heterocycloalkyl means a monocyclic or multicyclic ring system (which may be saturated or partially unsaturated), including fused and spiro rings, of about five to about 10 elements wherein one or more of the elements in the ring system is an element other than carbon and is selected from nitrogen, oxygen, silicon, or sulphur atoms. Heteroaryl means a five to about a 14-membered aromatic monocyclic or multicyclic hydrocarbon ring system, including fused and spiro rings, in which one or more of the elements in the ring system is an element other than carbon and is selected from nitrogen, oxygen, silicon, or sulphur and wherein an N atom may be in the form of an N-oxide. Arylcarbonyl means an aryl-CO— group in which the aryl group is as described herein. Heteroarylcarbonyl means a heteroaryl-CO— group in which the heteroaryl group is as described herein and heterocycloalkylcarbonyl means a heterocycloalkyl-CO— group in which the heterocycloalkyl group is as described herein. Aryloxycarbonyl means an ROC(O)— group where R is an aryl group as previously described. Heteroaryloxycarbonyl means an ROC(O)— group where R is a heteroaryl group as previously described. Heterocycloalkoxy means a heterocycloalkyl-O— group in which the heterocycloalkyl group is as previously described. Heterocycloalkoxycarbonyl means an ROC(O)— group where R is a heterocycloalkyl group as previously described. The term “acyl group” is intended to mean a group having the formula RCO—, wherein R is an alkyl group or an aryl group.

The term “alkenyl” refers to a straight or branched chain alkyl moiety having two or more carbon atoms (e.g., two to six carbon atoms, C₂₋₆ alkenyl) and having in addition one or more double bonds, of either E or Z stereochemistry where applicable. This term would include, for example, vinyl, 1-propenyl, 1- and 2-butenyl, 2-methyl-2-propenyl, etc.

The term “cycloalkenyl” refers to an alicyclic moiety having three or more carbon atoms (e.g., from three to six carbon atoms) and having in addition one or more double bonds. This term includes, for example, cyclopentenyl or cyclohexenyl.

The term “heterocycloalkenyl” refers to an alicyclic moiety having from three to six carbon atoms and one or more heteroatoms from the group N, O, S (or oxides thereof) and having in addition one or more double bonds. This term includes, for example, dihydropyranyl.

The term “halogen” means a halogen of the periodic table, such as fluorine, chlorine, bromine, or iodine.

The term “optionally substituted” means optionally substituted with one or more of the aforementioned groups (e.g., alkyl, aryl, heteroaryl, acyl, alkenyl, cycloalkyl, heterocycloalkyl, cycloalkenyl, heterocycloalkenyl, or halogen), at any available position or positions.

Examples of saturated alkyl groups include, but are not limited to, methyl, ethyl, N-propyl, isopropyl, N-butyl, tert-butyl, isobutyl, sec-butyl, N-pentyl, N-hexyl, N-heptyl, and N-octyl. An unsaturated alkyl group is one having one or more double or triple bonds. Unsaturated alkyl groups include, for example, ethenyl, propenyl, butenyl, hexenyl, vinyl, 2-propynyl, 2-isopentenyl, 2-butadienyl, ethynyl, 1-propynyl, 3-propynyl, and 3-butynyl. Cycloalkyl groups include, for example, cyclopropyl, cyclobutyl, cyclopentyl, cyclohexyl, 1-cyclohexenyl, 3-cyclohexenyl, cycloheptyl, indanyl, and tetrahydronaphthyl. Heterocycloalkyl groups include, for example, azetidinyl, indolinyl, 1-piperidinyl, 2-piperidinyl, 3-piperidinyl, 3-morpholinyl, 4-morpholinyl, tetrahydrofuran-2-yl, tetrahydrofuran-3-yl, tetrahydrothien-2-yl, tetrahydrothien-3-yl, tetrahydroquinolinyl, 1-piperazinyl, 2-piperazinyl, and 1,4-diazabicyclooctane. Aryl groups include, for example, phenyl, indenyl, biphenyl, 1-naphthyl, 2-naphthyl, anthracenyl, and phenanthracenyl. Heteroaryl groups include, for example, 1-pyrrolyl, 2-pyrrolyl, 3-pyrrolyl, furyl, thienyl, imidazolyl, oxazolyl, thiazolyl, pyrazolyl, pyridyl, indolyl, quinolinyl, isoquinolinyl, thiophenyl, benzoquinolinyl, carbazolyl, and diazaphenanthrenyl.

Specifically, “alkyl” can include, for example, methyl, ethyl, propyl, isopropyl, butyl, iso-butyl, sec-butyl, pentyl, 3-pentyl, hexyl, heptyl, octyl, nonyl, decyl, undecyl, dodecyl, tridecyl, tetradecyl or pentadecyl; “alkenyl” can include vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl; 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, 1-tridecenyl, 2-tridecenyl, 3-tridecenyl, 4-tridecenyl, 5-tridecenyl, 6-tridecenyl, 7-tridecenyl, 8-tridecenyl, 9-tridecenyl, 10-tridecenyl, 11-tridecenyl, 12-tridecenyl, 1-tetradecenyl, 2-tetradecenyl, 3-tetradecenyl, 4-tetradecenyl, 5-tetradecenyl, 6-tetradecenyl, 7-tetradecenyl, 8-tetradecenyl, 9-tetradecenyl, 10-tetradecenyl, 11-tetradecenyl, 12-tetradecenyl, 13-tetradecenyl, 1-pentadecenyl, 2-pentadecenyl, 3-pentadecenyl, 4-pentadecenyl, 5-pentadecenyl, 6-pentadecenyl, 7-pentadecenyl, 8-pentadecenyl, 9-pentadecenyl, 10-pentadecenyl, 11-pentadecenyl, 12-pentadecenyl, 13-pentadecenyl, or 14-pentadecenyl; “alkoxy” can include methoxy, ethoxy, propoxy, isopropoxy, butoxy, iso-butoxy, sec-butoxy, pentoxy, 3-pentoxy, hexoxy, heptyloxy, octyloxy, nonyloxy, decyloxy, undecyloxy, dodecyloxy, tridecyloxy, tetradecyloxy, or pentadecyloxy; “alkanoyl” can include acetyl, propanoyl, butanoyl, pentanoyl, hexanoyl, heptanoyl, octanoyl, nonanoyl, decanoyl, undecanoyl, dodecanoyl, tridecanoyl, tetradecanoyl, or pentadecanoyl; “cycloalkyl” can include cyclopropyl, cyclobutyl, cyclopentyl, cyclohexyl, cycloheptyl, or cyclooctyl, for example; “aryl” can include phenyl, indenyl, 5,6,7,8-tetrahydronaphthyl, or naphthyl, for example; and “heteroaryl” can include furyl, imidazolyl, tetrazolyl, pyridyl, (or its N-oxide), thienyl, pyrimidinyl (or its N-oxide), indolyl, or quinolyl (or its N-oxide), for example.

The farnesylamine derivatives of the present invention include all hydrates and salts of farnesylamine derivatives of the invention (e.g., compounds I-VI), or of their analogs, that can be prepared by those of skill in the art. Under conditions where the compounds of the present invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of farnesylamine derivatives may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “analogs” refers to compounds which are substantially the same as another compound but which may have been modified by, for example, adding side groups, oxidation or reduction of the parent structure. Analogs of the farnesylamine derivatives of the invention can be readily prepared using commonly known standard reactions. These standard reactions include, but are not limited to, hydrogenation, alkylation, acetylation, and acidification reactions. Chemical modifications can be accomplished by those skilled in the art by protecting all functional groups present in the molecule and deprotecting them after carrying out the desired reactions using standard procedures known in the scientific literature (Greene et al. 1999; Honda et al. 1997; Honda et al. 1998; Konoike et al. 1997; Honda et al. 2000; each of which are hereby incorporated herein by reference in their entirety). Analogs exhibiting the desired biological activity (such as induction of apoptosis, cytotoxicity, cytostaticity, induction of cell cycle arrest, anti-angiogenic properties, etc.) can be identified or confirmed using cellular assays or other in vitro or in vivo assays. For example, assays that detect inhibition of farnesylation of oncogenic proteins, G₂/M cell cycle arrest, angiogenesis, and/or reduction of tumor growth may be utilized.

It will be appreciated that the farnesylamine derivatives of the invention can contain one or more asymmetrically substituted carbon atoms (i.e., carbon centers). The presence of one or more of the asymmetric centers in an analog of the invention, can give rise to stereoisomers, and in each case, the invention is to be understood to extend to all such stereoisomers, including enantiomers and diastereomers, and mixtures including racemic mixtures thereof.

The farnesylamine derivatives of the invention are useful for various non-therapeutic and therapeutic purposes. The farnesylamine derivatives may be used for reducing aberrant cell growth in animals and humans. Because of such anti-proliferative properties of the compounds, they are useful in reducing unwanted cell growth in a wide variety of settings including in vitro and in vivo. In addition to their use in treatment methods, the farnesylamine derivatives of the invention are useful as agents for investigating the role of ras in cellular metabolism, and controlling ras-mediated malignant or non-malignant cell growth in vitro or in vivo. They are also useful as standards and for teaching demonstrations.

Therapeutic application of the farnesylamine derivatives and compositions containing them can be accomplished by any suitable therapeutic method and technique presently or prospectively known to those skilled in the art. Further, the farnesylamine derivatives of the invention have use as starting materials or intermediates for the preparation of other useful compounds and compositions.

Farnesylamine derivatives of the invention, and compositions comprising a farnesyl derivative, may be locally administered at one or more anatomical sites, such as sites of unwanted cell growth (such as a tumor site or benign skin growth, e.g., injected or topically applied to the tumor or skin growth) or sites of fungal infection, optionally in combination with a pharmaceutically acceptable carrier such as an inert diluent. Farnesylamine derivatives of the invention, and compositions comprising a farnesyl derivative, may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active agent (e.g., compounds I-VI, or pharmaceutically acceptable salts or analogs thereof) may also be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The natural antifungal properties of farnesylamine derivatives may be relied upon to prevent the action of microorganisms. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by, incorporating the farnesylamine derivatives in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the farnesylamine derivatives may be applied in pure-form, i.e., when they are liquids. However, it will generally be desirable to administer them topically to the skin as compositions, in combination with a dermatologically acceptable carrier, which may be a solid or a liquid.

The farnesylamine derivatives of the subject invention can be applied topically to a subject's skin to reduce the size (and may include complete removal) of malignant or benign growths, or to treat an infection site. The farnesylamine derivatives of the invention can be applied directly to the growth or infection site. Preferably, the farnesylamine derivative is applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the farnesylamine derivative can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty, alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the farnesylamine derivatives to the skin are disclosed in U.S. Pat. No. 4,608,392; U.S. Pat. No. 4,992,478; U.S. Pat. No. 4,559,157; and U.S. Pat. No. 4,820,508.

Useful dosages of the pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Accordingly, the present invention includes a pharmaceutical composition comprising a farnesylamine derivative of the invention in combination with a pharmaceutically acceptable carrier. Pharmaceutical compositions adapted for oral, topical or parenteral administration, comprising an amount of farnesylamine derivative constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Depending upon the disorder or disease condition to be treated, a suitable dose(s) may be that amount that will reduce proliferation or growth of the target cell(s). In the context of cancer, a suitable dose(s) is that which will result in a concentration of the active agent in cancer tissue, such as a malignant tumor, which is known to achieve the desired response. The preferred dosage is the amount which results in maximum inhibition of cancer cell growth, without unmanageable side effects. Administration of a farnesylamine derivative can be continuous or at distinct intervals, as can be determined by a person of ordinary skill in the art.

To provide for the administration of such dosages for the desired therapeutic treatment, in some embodiments, pharmaceutical compositions of the invention can comprise between about 0.1% and 45%, and especially, 1 and 15%, by weight of the total of one or more of the farnesylamine derivatives based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Mammalian species which benefit from the disclosed methods include, but are not limited to, primates, such as apes, chimpanzees, orangutans, humans, monkeys;

domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cells of such human and non-human species.

Patients in need of treatment using the methods of the present invention can be identified using standard techniques known to those in the medical or veterinary professions, as appropriate.

As used herein, the terms “cancer” and “cancerous” refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas. Examples of cancer types that may potentially be treated using the farnesylamine derivatives of the present invention are also listed in Table 1.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult |
| Acute Myeloid Leukemia, Adult | (Primary) |
| Acute Myeloid Leukemia, Childhood | Hepatocellular (Liver) Cancer, Childhood |
| Adrenocortical Carcinoma | (Primary) |
| Adrenocortical Carcinoma, Childhood | Hodgkin's Lymphoma, Adult |
| AIDS-Related Cancers | Hodgkin's Lymphoma, Childhood |
| AIDS-Related Lymphoma | Hodgkin's Lymphoma During Pregnancy |
| Anal Cancer | Hypopharyngeal Cancer |
| Astrocytoma, Childhood Cerebellar | Hypothalamic and Visual Pathway Glioma, |
| Astrocytoma, Childhood Cerebral | Childhood |
| Basal Cell Carcinoma | Intraocular Melanoma |
| Bile Duct Cancer, Extrahepatic | Islet Cell Carcinoma (Endocrine Pancreas) |
| Bladder Cancer | Kaposi's Sarcoma |
| Bladder Cancer, Childhood | Kidney (Renal Cell) Cancer |
| Bone Cancer, Osteosarcoma/Malignant | Kidney Cancer, Childhood |
| Fibrous Histiocytoma | Laryngeal Cancer |
| Brain Stem Glioma, Childhood | Laryngeal Cancer, Childhood |
| Brain Tumor, Adult | Leukemia, Acute Lymphoblastic, Adult |
| Brain Tumor, Brain Stem Glioma, | Leukemia, Acute Lymphoblastic, Childhood |
| Childhood | Leukemia, Acute Myeloid, Adult |
| Brain Tumor, Cerebellar Astrocytoma, | Leukemia, Acute Myeloid, Childhood |
| Childhood | Leukemia, Chronic Lymphocytic |
| Brain Tumor, Cerebral | Leukemia, Chronic Myelogenous |
| Astrocytoma/Malignant Glioma, | Leukemia, Hairy Cell |
| Childhood | Lip and Oral Cavity Cancer |
| Brain Tumor, Ependymoma, Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial Primitive | Lung Cancer, Small Cell |
| Neuroectodermal Tumors, Childhood | Lymphoma, AIDS-Related |
| Brain Tumor, Visual Pathway and | Lymphoma, Burkitt's |
| Hypothalamic Glioma, Childhood | Lymphoma, Cutaneous T-Cell, see Mycosis |
| Brain Tumor, Childhood | Fungoides and Sézary Syndrome |
| Breast Cancer | Lymphoma, Hodgkin's, Adult |
| Breast Cancer, Childhood | Lymphoma, Hodgkin's, Childhood |
| Breast Cancer, Male | Lymphoma, Hodgkin's During Pregnancy |
| Bronchial Adenomas/Carcinoids, | Lymphoma, Non-Hodgkin's, Adult |
| Childhood | Lymphoma, Non-Hodgkin's, Childhood |
| Burkitt's Lymphoma | Lymphoma, Non-Hodgkin's During |
| Carcinoid Tumor, Childhood | Pregnancy |
| Carcinoid Tumor, Gastrointestinal | Lymphoma, Primary Central Nervous System |
| Carcinoma of Unknown Primary | Macroglobulinemia, Waldenström's |
| Central Nervous System Lymphoma, | Malignant Fibrous Histiocytoma of |
| Primary | Bone/Osteosarcoma |
| Cerebellar Astrocytoma, Childhood | Medulloblastoma, Childhood |
| Cerebral Astrocytoma/Malignant | Melanoma |
| Glioma, Childhood | Melanoma, Intraocular (Eye) |
| Cervical Cancer | Merkel Cell Carcinoma |
| Childhood Cancers | Mesothelioma, Adult Malignant |
| Chronic Lymphocytic Leukemia | Mesothelioma, Childhood |
| Chronic Myelogenous Leukemia | Metastatic Squamous Neck Cancer with |
| Chronic Myeloproliferative Disorders | Occult Primary |
| Colon Cancer | Multiple Endocrine Neoplasia Syndrome, |
| Colorectal Cancer, Childhood | Childhood |
| Cutaneous T-Cell Lymphoma, see | Multiple Myeloma/Plasma Cell Neoplasm |
| Mycosis Fungoides and Sézary | Mycosis Fungoides |
| Syndrome | Myelodysplastic Syndromes |
| Endometrial Cancer | Myelodysplastic/Myeloproliferative Diseases |
| Ependymoma, Childhood | Myelogenous Leukemia, Chronic |
| Esophageal Cancer | Myeloid Leukemia, Adult Acute |
| Esophageal Cancer, Childhood | Myeloid Leukemia, Childhood Acute |
| Ewing's Family of Tumors | Myeloma, Multiple |
| Extracranial Germ Cell Tumor, | Myeloproliferative Disorders, Chronic |
| Childhood | Nasal Cavity and Paranasal Sinus Cancer |
| Extragonadal Germ Cell Tumor | Nasopharyngeal Cancer |
| Extrahepatic Bile Duct Cancer | Nasopharyngeal Cancer, Childhood |
| Eye Cancer, Intraocular Melanoma | Neuroblastoma |
| Eye Cancer, Retinoblastoma | Non-Hodgkin's Lymphoma, Adult |
| Gallbladder Cancer | Non-Hodgkin's Lymphoma, Childhood |
| Gastric (Stomach) Cancer | Non-Hodgkin's Lymphoma During Pregnancy |
| Gastric (Stomach) Cancer, Childhood | Non-Small Cell Lung Cancer |
| Gastrointestinal Carcinoid Tumor | Oral Cancer, Childhood |
| Germ Cell Tumor, Extracranial, | Oral Cavity Cancer, Lip and |
| Childhood | Oropharyngeal Cancer |
| Germ Cell Tumor, Extragonadal | Osteosarcoma/Malignant Fibrous |
| Germ Cell Tumor, Ovarian | Histiocytoma of Bone |
| Gestational Trophoblastic Tumor | Ovarian Cancer, Childhood |
| Glioma, Adult | Ovarian Epithelial Cancer |
| Glioma, Childhood Brain Stem | Ovarian Germ Cell Tumor |
| Glioma, Childhood Cerebral | Ovarian Low Malignant Potential Tumor |
| Astrocytoma | Pancreatic Cancer |
| Glioma, Childhood Visual Pathway and | Pancreatic Cancer, Childhood |
| Hypothalamic | Pancreatic Cancer, Islet Cell |
| Skin Cancer (Melanoma) | Paranasal Sinus and Nasal Cavity Cancer |
| Skin Carcinoma, Merkel Cell | Parathyroid Cancer |
| Small Cell Lung Cancer | Penile Cancer |
| Small Intestine Cancer | Pheochromocytoma |
| Soft Tissue Sarcoma, Adult | Pineoblastoma and Supratentorial Primitive |
| Soft Tissue Sarcoma, Childhood | Neuroectodermal Tumors, Childhood |
| Squamous Cell Carcinoma, see Skin | Pituitary Tumor |
| Cancer (non-Melanoma) | Plasma Cell Neoplasm/Multiple Myeloma |
| Squamous Neck Cancer with Occult | Pleuropulmonary Blastoma |
| Primary, Metastatic | Pregnancy and Breast Cancer |
| Stomach (Gastric) Cancer | Pregnancy and Hodgkin's Lymphoma |
| Stomach (Gastric) Cancer, Childhood | Pregnancy and Non-Hodgkin's Lymphoma |
| Supratentorial Primitive | Primary Central Nervous System Lymphoma |
| Neuroectodermal Tumors, Childhood | Prostate Cancer |
| T-Cell Lymphoma, Cutaneous, see | Rectal Cancer |
| Mycosis Fungoides and Sézary | Renal Cell (Kidney) Cancer |
| Syndrome | Renal Cell (Kidney) Cancer, Childhood |
| Testicular Cancer | Renal Pelvis and Ureter, Transitional Cell |
| Thymoma, Childhood | Cancer |
| Thymoma and Thymic Carcinoma | Retinoblastoma |
| Thyroid Cancer | Rhabdomyosarcoma, Childhood |
| Thyroid Cancer, Childhood | Salivary Gland Cancer |
| Transitional Cell Cancer of the Renal | Salivary Gland Cancer, Childhood |
| Pelvis and Ureter | Sarcoma, Ewing's Family of Tumors |
| Trophoblastic Tumor, Gestational | Sarcoma, Kaposi's |
| Unknown Primary Site, Carcinoma of, | Sarcoma, Soft Tissue, Adult |
| Adult | Sarcoma, Soft Tissue, Childhood |
| Unknown Primary Site, Cancer of, | Sarcoma, Uterine |
| Childhood | Sezary Syndrome |
| Unusual Cancers of Childhood | Skin Cancer (non-Melanoma) |
| Ureter and Renal Pelvis, Transitional | Skin Cancer, Childhood |
| Cell Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. The treatment methods of the invention can be utilized for early, middle, or late stage disease, and acute or chronic disease. In some embodiments, the tumor is characterized as one having a K-ras mutation.

In methods of the subject invention, the farnesylamine derivative can be administered to a patient by itself, or co-administered with another agent such as another farnesylamine derivative, or a different agent. Co-administration can be carried out simultaneously (in the same or separate formulations) or consecutively. Furthermore, in certain embodiments of a method of the subject invention, farnesylamine derivatives can be administered to a patient as adjuvant therapy. For example, farnesylamine derivatives can be administered to a patient in conjunction with chemotherapy.

Thus, the farnesylamine derivatives of the invention, whether administered separately, or as a pharmaceutical composition, can include various other components as additives. Examples of acceptable components or adjuncts which can be employed in relevant circumstances include antioxidants, free radical scavenging agents, peptides, growth factors, antibiotics, bacteriostatic agents, immunosuppressives, anticoagulants, buffering agents, anti-inflammatory agents, anti-angiogenics, anti-pyretics, time-release binders, anesthetics, steroids, and corticosteroids. Such components can provide additional therapeutic benefit, act to affect the therapeutic action of the farnesylamine derivatives, or act towards preventing any potential side effects which may be posed as a result of administration of the farnesylamine derivatives. The farnesylamine derivatives of the subject invention can be conjugated to a therapeutic agent, as well.

Additional agents that can be co-administered to target cells in vitro or in vivo, such as in a patient, in the same or as a separate formulation, include those that modify a given biological response, such as immunomodulators. For example, proteins such as tumor necrosis factor (TNF), interferon (such as alpha-interferon and beta-interferon), nerve growth factor (NGF), platelet derived growth factor (PDGF), and tissue plasminogen activator can be administered. Biological response modifiers, such as lymphokines, interleukins (such as interleukin-1 (IL-1), interleukin-2 (IL-2), and interleukin-6 (IL-6)), granulocyte macrophage colony stimulating factor (GM-CSF), granulocyte colony stimulating factor (G-CSF), or other growth factors can be administered. In one embodiment, the methods and compositions of the invention incorporate one or more agents selected from the group consisting of anti-cancer agents, cytotoxic agents, chemotherapeutic agents, anti-signaling agents, and anti-angiogenic agents.

The subject invention also concerns kits comprising one or more farnesyl derivatives of the invention, or a composition comprising a farnesyl derivative, or an analog or salt of the foregoing, in one or more containers. In one embodiment, the farnesyl derivative is one or more of N-farnesylphthalimide (compound I), N-methyl-N′-farnesylphthalamide (compound II), N,N′-bisfarnesylphthalamide (compound III), farnesylamine acetate (compound IV), farnesylamine propionate (compound V), and farnesylamine palmitate (compound VI); or a pharmaceutically acceptable salt or analog of any of the foregoing. Kits of the invention can optionally include pharmaceutically acceptable carriers and/or diluents. In one embodiment, a kit of the invention includes one or more other components, adjuncts, or adjuvants as described herein. In another embodiment, a kit includes agents that modify a given biological response, such as those agents described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a farnesyl derivative of the invention is provided in the kit as a solid, such as a tablet, pill, or powder form. In another embodiment, a farnesyl derivative of the invention is provided in the kit as a liquid or solution. In one embodiment, the kit comprises an ampoule or syringe containing a farnesyl derivative of the invention in liquid or solution form.

A further aspect of the subject invention provides a process for synthesizing farnesylamine derivatives of the invention.

The method for producing N-farnesylphthalimide (compound I) includes farnesyl alcohol activation with DIAD/Ph₃P and its treatment with phthalimide. In one embodiment, the method involves dissolving farnesyl alcohol in anhydrous THF with (Ph)₃P and phthamide, and adding DIAD dropwise to the reaction mixture. Once the THF is evaporated, the reaction mixture is then extracted with hexanes; column chromatographic separation of the crude mixture yields compound I.

The method for producing N-methyl-N′-farnesylphthalamide (compound II) and/or N,N′-bisfarnesylphthalamide (compound III) includes treatment of compound I with methyl amine. In one embodiment, compound I is dissolved in ethanol with methylamine and the reaction mixture is refluxed. Ethanol is then evaporated under reduced pressure and compounds II and III are separated by column chromatography by eluting with 5% methanol/dichloromethane.

The method for producing farnesylamine acetate (compound IV), farnesylamine propionate (compound V), and/or farnesylamine palmitate (compound VI) includes acylation of franesyl amine with acid anhydride. In one embodiment, farnesylamine is dissolved in THF with a catalytic amount of dimethylaminopyridine (DMAP), and acid anhydride is added (acetic anhydride for compound IV, propionic anhydride for compound V, and palmitic anhydride for compound VI). THF is then evaporated and the residue dissolved in ethyl acetate and extracted successively with two portions of dilute HCl, sodium bicarbonate solution, and water. The organic layer is then dried with NaSO₄. Finally, the organic layer is evaporated under reduced pressure to yield compounds IV, V, and VI in quantitative yields.

A scientist skilled in the art of organic synthesis and purification can easily adapt the foregoing methods and substitute a variety of solvents and stationary phases for those described in the preferred embodiments of the invention. For example, solid-liquid extraction or liquid-liquid extraction may be used to obtain the isolated farnesylamine derivative. In addition to chromatography, methods such as crystallization and partitioning can also be used to purify the desired compounds. See, for example, Brown 1956; McCabe et al. 1956; and Perry et al. 1997, which are incorporated herein by reference in their entirety.

There is no particular limitation as to the method for extracting the farnesylamine derivatives. For example, extraction with various solvents or supercritical fluid extraction is applicable. Examples of suitable solvents include aqueous media such as water, inorganic salt aqueous solution and buffer solutions, and organic solvents such as alcohol, hexane, toluene, petroleum ether, benzene, ethyl acetate, chloroform, dichloromethane, 1,1,2-trichloroethane, di-methylsulfoxide, and acetone, among which alcohol is preferred. Water can be water, distilled water, deionized water, or pure water. Examples of buffer solution that may be used include phosphate buffer and citrate buffer.

Examples of the alcohol that may be used include monohydric alcohols such as methanol, ethanol, propanol and butanol, and multi-hydric alcohols such as propylene glycol and glycerol, among which a monohydric alcohol is preferred, and particularly ethanol is preferred. These solvents may be used alone or as a mixture. As the mixed solvent, water-containing alcohols are preferred. Water-containing monovalent alcohols are more preferred, and water-containing ethanol is particularly preferred.

There is no particular limitation as to the apparatus used for extraction, and a vessel designed for efficient extraction, a stirrer, a reflux condenser, a Soxhlet extractor, a homogenizer, a shaker, a supersonic generator, etc., may be used. The liquid extract may be treated by means of various solid-liquid separation such as sedimentation, cake filtration, clear filtration, centrifugal filtration, centrifugal sedimentation, compression separation or filter press.

### DEFINITIONS

As used herein, the terms “treat” or “treatment” refer to both therapeutic treatment and prophylactic or preventative measures, wherein the object is to prevent or slow down (lessen) an undesired physiological change or disorder, such as the development or spread of cancer or other proliferation disorder or infection. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with a farnesylamine derivative may include reduction of undesirable cell proliferation, and/or induction of apoptosis and cytotoxicity. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the patient may be identified (e.g., diagnosed) as one suffering from the disease or condition (e.g., proliferation disorder, fungal infection) prior to administration of the farnesylamine derivative of the invention.

As used herein, the term “(therapeutically) effective amount” refers to an amount of the farnesylamine derivative or other agent (e.g., a drug) effective to treat a disease or disorder in a mammal. In the case of cancer or other proliferation disorder, the therapeutically effective amount of the agent may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the farnesylamine derivative prevents growth and/or kill existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the farnesylamine derivative refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

The terms “cell” and “cells” are used interchangeably herein and are intended to include either a single cell or a plurality of cells, in vitro or in vivo, unless otherwise specified.

As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY). In some embodiments, the anti-cancer agent is a ras antagonist.

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. In a preferred embodiment, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “a compound” includes more than one such compound. A reference to “a farnesylamine derivative” includes more than one such derivative, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook et al. 1989; Glover 1985; Perbal 1984; Colowick et al.; Hames et al. 1984; Miller et al. 1987; Scopes, Protein Purification: Principles and Practice (2nd ed., Springer-Verlag); and McPherson et al. 1991), each of which are incorporated herein by reference in their entirety. Following are examples that illustrate materials, methods, and procedures for practicing the invention. The examples are illustrative and should not be construed as limiting.

### Example 1

### Synthesis of N′ Methyl N′ Farnesyl Phthalimide (Compound I)

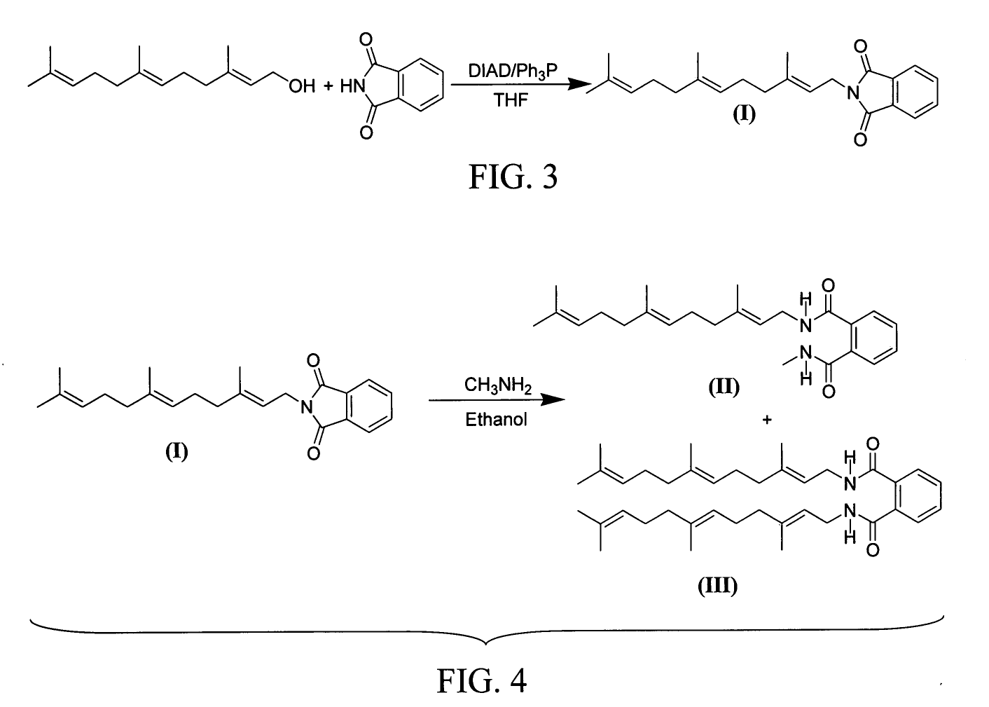

Farnesyl alcohol was dissolved in THF with (Ph)₃P and phthamide. Next, DIAD was added drop wise to the reaction mixture. After 5 hours, THF was evaporated and the reaction mixture was extracted with hexanes to yield (I), as shown by the scheme in FIG. 3. The compound was further purified by column chromatography by eluting with 3% ethyl acetate/hexanes. Yield (88%).

### Example 2

### Synthesis of N′N′ Difarnesylamine Acetate (Compound II) and N′N′ Difarnesylamine Acetate (Compound III)

Compound (I) was dissolved in ethanol with ½ mole equivalent of methylamine. The reaction was reflux 4 hours. Next, ethanol was evaporated under reduced pressure, and the compounds (II) and (III) were separated by column chromatography by eluting with 5% methanol/dichloromethane, as shown by the scheme of FIG. 4.

### Example 3

### Synthesis of Farnesylamine Acetate (Compound IV), Farnesylamine Propionate (Compound V), and Farnesylamine Palmitate (Compound VI)

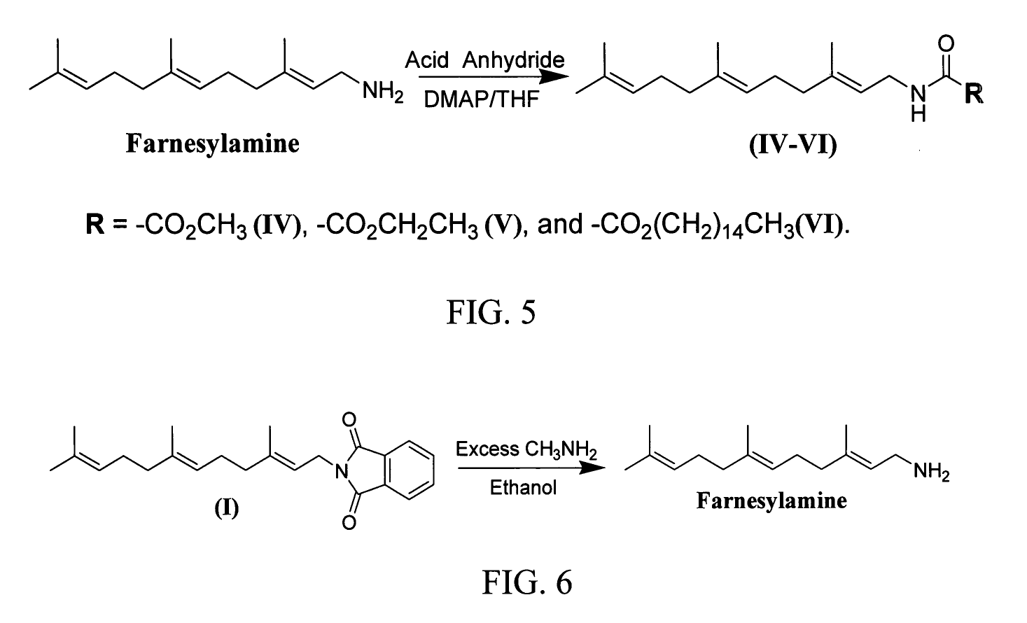

Farnesylamine was dissolved in THF with catalytic amount of dimethylaminopyridine (DMAP). Next, acid anhydride was added (acetic anhydride (IV), propionic anhydride (V), and palmitic anhydride (VI)). When the reaction was completed, THF was evaporated and residue dissolved in ethyl acetate and extracted successively with two portions of dilute HCl, sodium bicarbonate solution, and water. Next, the organic layer was dried with NaSO₄. Finally, the organic layer was evaporated under reduced pressure to yield compounds (IV), (V), and (VI) in quantitative yields, as shown by the scheme of FIG. 5.

Farnesylamine was prepared from the reaction of compound (I) with excess quantity of methylamine in ethanol, as shown by the scheme of FIG. 6. Farnesylamine was obtained quantitatively.

Farnesylamine derivatives of the invention are very potent inhibitors of farnesylation of oncogenic proteins, thereby inhibiting tumor growth at lower IC₅₀ values.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### REFERENCES

U.S. Pat. No. 4,559,157
U.S. Pat. No. 4,608,392
U.S. Pat. No. 4,820,508
U.S. Pat. No. 4,938,949
U.S. Pat. No. 4,992,478
U.S. Pat. No. 5,167,649
U.S. Pat. No. 6,462,086
Barbacid, M. Ann. Rev. Biochem., 1987, 56:779-827
Brown G. G., Unit Operations, John Wiley & Sons, 1956
Colowick, S, and Kaplan, N. (Eds.), the series, Methods In Enzymology, Academic Press, Inc.
Cox, A. D. and Der, C. J. Biochem Biophys Acta, 1997, 1333:F51-F71
Gana-Weisz, M. et al. Biochem. Biophys. Res. Commn., 1997, 239:900-904
Gibbs, J. B. et al. Cell, 1994, 77:175-178
Glover, D. N. (Ed.), 1985, DNA Cloning, Vols. I and II
Greene, T. W. and Wuts, P. G. M. “Protective Groups in Organic Synthesis” John Wiley & Sons, Inc. New York. 3rd Ed. pg. 819, 1999
Hames et al. (Eds.), 1984, Transcription and Translation
Honda, T. et al. Bioorg. Med. Chem. Lett., 1997, 7:1623-1628
Honda, T. et al. Bioorg. Med. Chem. Lett., 1998, 8:2711-2714
Honda, T. et al. J. Med. Chem., 2000, 43:4233-4246
Kato, K. et al. Proc. Nat. Acad. Sci., 1992, 89:6403-6407
Kiaris, H. and Spandidos, D. Int J Oncol., 1995, 7:413-421
Kloog, et al., Exp. Opin. Invest. Drugs, 1999, 8(12):2121-2140
Konoike, T. et al. J. Org. Chem., 1997, 62:960-966
Kothapalli, R. et al. Biochem. Pharmacol., 1994, 47:1909-1916
Kothapalli, R. et al. Lipids, 1993, 28:969-973
Maltese, W. A. and Robishaw, J. D. J. Biol. Chem., 1990a, 265:18071-18074
Maltese, W. A. and Sheridan, K. M. J. Biol. Chem., 1990b, 265:17883-17890
Maltese, W. A. et al. J. Biol. Chem., 1990c, 265:2148-2155
Maltese, W. A. FASEB 1, 1990, 4:3319-3328
Martin, E. W., 1995, Easton Pa., Mack Publishing Company, 19^{th} ed.
McCabe, W. L. and J. C. Smith, Unit Operations of Chemical Engineering, McGraw-Hill, 1956
McPherson et al. (Eds.), 1991, PCR: A Practical Approach, IRL Press
Meigs, T. E. et al. Exp Cell Res, 1995, 219:461-470
Miller, J. H. et al. (Eds.), 1987, Gene Transfer Vectors For Mammalian Cells, Cold Spring Harbor Laboratory, Cold Spring Harbor, N.Y.
Overhand et al. Bioorg Med Chem Lett, 1997, 7:2435, 2440
Perbal, B., A Practical Guide to Molecular Cloning (1984)
Perry, R. H., and D. Green, Perry's Chemical Engineers' Handbook, 7^{th} Edition, McGraw-Hill, 1997
Sambrook, Fritsch & Maniatis, Molecular Cloning: A Laboratory Manual, Second Edition (1989)
Sebti, S. M. and Hamilton, A. D. Pharmacol Ther, 1997, 74:103-114
Tahir, S. K. et al. Eur Journal Cancer, 2000, 36:1161-1170
Ura, H. et al. Mole carcinogen, 1998, 21:93-99

1. A compound of formula A or formula B: 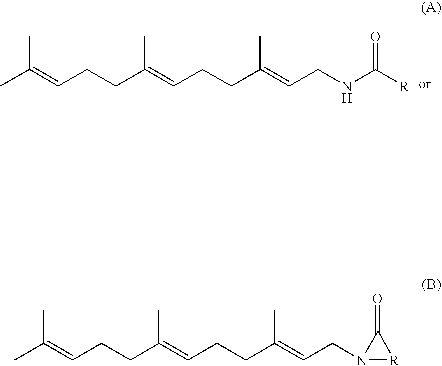
wherein R is one or more aliphatic or aromatic functional groups or substituents optionally substituted with any substituent, or a pharmaceutically acceptable salt or analog of a compound of formula A and/or formula B.

2. The compound of claim 1, wherein R is one or more of H, alkyl, alkoxy, cycloalkyl, cycloalkoxy, heterocycloalkyl, alkyl-NHC(O)—, acyl, aryl, aryl-NHC(O)—, aryloxy, alkylcarbonyl, alkoxycarbonyl, cycloalkylcarbonyl, cycloalkoxycarbonyl, heteroalkyl, heteroaryl, arylcarbonyl, heteroarylcarbonyl, heterocycloalkylcarbonyl, aryloxycarbonyl, heteroaryloxycarbonyl, heterocycloalkoxy, and/or heterocycloalkoxycarbonyl.

3. The compound of claim 1, wherein R is optionally substituted with one or more of any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

4. The compound of claim 1, wherein R is an alkyl, optionally substituted with any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

5. The compound of claim 4, wherein R is methyl, ethyl, butyl, pentyl, hexyl, heptyl, octyl, or nonyl.

6. The compound of claim 1, wherein R is aryl, optionally substituted with any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

7. The compound of claim 6, wherein R is phenyl.

8. The compound of claim 1, wherein the compound is N-farnesylphthalimide (compound I), N-methyl-N′-farnesylphthalamide (compound II), N,N′-bisfarnesylphthalamide (compound III), farnesylamine acetate (compound IV), farnesylamine propionate (compound V), farnesylamine palmitate (compound VI), or a pharmaceutically acceptable salt or analog of any of the foregoing.

9. The compound of claim 1, wherein the compound has the structure:

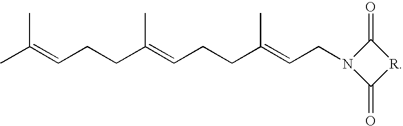

10. A composition comprising a compound of formula A or formula B: 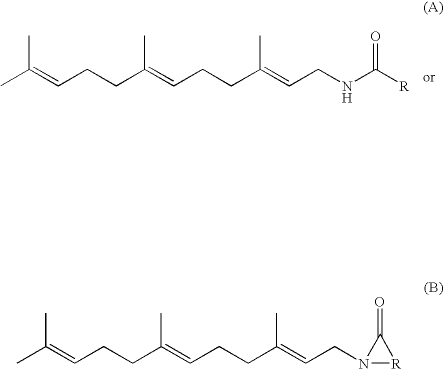
wherein R is one or more aliphatic or aromatic functional groups or substituents optionally substituted with any substituent, or a pharmaceutically acceptable salt or analog of a compound of formula A and/or formula B; and a pharmaceutically acceptable carrier.

11. The composition of claim 10, further comprising an additional anti-cancer agent or anti-fungal agent; and/or
further comprising an agent selected from the group consisting of an antioxidant, free radical scavenging agent, peptide, growth factor, antibiotic, bacteriostatic agent, immunosuppressive, anticoagulant, buffering agent, anti-inflammatory agent, anti-angiogenic agent, anti-pyretic, time-release binder, anesthetic, steroid, and corticosteroid; and/or
further comprising a ras antagonist.

12-13. (canceled)

14. A method for treating a disorder in a patient, comprising administering at least one compound of formula A and/or formula B, or a composition comprising a compound of formula A and/or formula B and a pharmaceutically acceptable carrier: 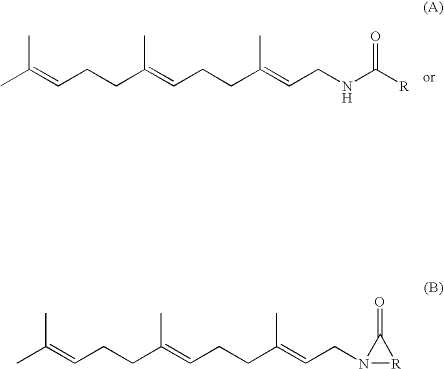
wherein R is one or more aliphatic or aromatic functional groups or substituents optionally substituted with any substituent, or a pharmaceutically acceptable salt or analog of a compound of formula A and/or formula B, to a patient in need of treatment, wherein the disorder is a proliferation disorder or fungal infection.

15. The method of claim 14, wherein R is one or more of H, alkyl, alkoxy, cycloalkyl, cycloalkoxy, heterocycloalkyl, alkyl-NHC(O)—, acyl, aryl, aryl-NHC(O)—, aryloxy, alkylcarbonyl, alkoxycarbonyl, cycloalkylcarbonyl, cycloalkoxycarbonyl, heteroalkyl, heteroaryl, arylcarbonyl, heteroarylcarbonyl, heterocycloalkylcarbonyl, aryloxycarbonyl, heteroaryloxycarbonyl, heterocycloalkoxy, and/or heterocycloalkoxycarbonyl.

16. The method according to claim 14, wherein R is optionally substituted with one or more of any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

17. The method of claim 14, wherein R is an alkyl, optionally substituted with any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

18. The method of claim 17, wherein R is methyl, ethyl, butyl, pentyl, hexyl, heptyl, octyl, or nonyl.

19. The method of claim 14, wherein R is aryl, optionally substituted with any halogen, —COOH, —OH, —NO₂, —NH₂, —N-alkyl, alkyl, alkyl-NHC(O)—, alkoxy, cycloalkyl, cycloalkoxy, aryl, aryl-NHC(O)—, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, and/or heterocycloalkoxycarbonyl.

20. The method of claim 19, wherein R is phenyl.

21. The method of claim 14, wherein the at least one compound is selected from N-farnesylphthalimide (compound I), N-methyl-N′-farnesylphthalamide (compound II), N,N-bisfarnesylphthalamide (compound III), farnesylamine acetate (compound IV), farnesylamine propionate (compound V), or farnesylamine palmitate (compound VI), or a pharmaceutically acceptable salt or analog of any of the foregoing, to the patient.

22. The method of claim 14, wherein the compound has the structure:

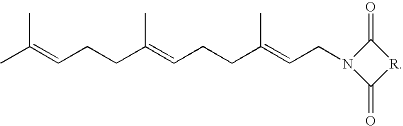

23. The method of any of claim 14, wherein the disorder is a proliferation disorder, and wherein the proliferation disorder is cancer.

24. The method of claim 14, wherein the at least one compound is administered locally at the site of a tumor.

25. The method of claim 14, wherein the disorder is a proliferation disorder, wherein the proliferation disorder is cancer, and wherein the patient is suffering from a tumor and the at least one compound inhibits growth of the tumor.

26. The method of claim 14, wherein the disorder is a proliferation disorder, and the proliferation disorder is a non-malignant disease characterized by Ras mediated proliferation of cells.

27. (canceled)

28. The method of claim 14, wherein the patient is not suffering from the disorder but is at risk of developing the disorder, and wherein the at least one compound is administered to delay onset of the disorder.

29. The method of claim 14, wherein the route of administration is selected from the group consisting of enteral, parenteral, intravenous, intramuscular, oral, subcutaneous, topical, and intra-nasal.

30. The method of claim 14, wherein the patient is human, or a non-human mammal.

31. (canceled)

32. The method of claim 14, further comprising identifying the patient as one suffering from the disorder.

33. A method for disinfecting a substrate, comprising applying or contacting the substrate with at least one compound of formula A and/or formula B, or a composition comprising a compound of formula A and/or formula B and a pharmaceutically acceptable carrier: 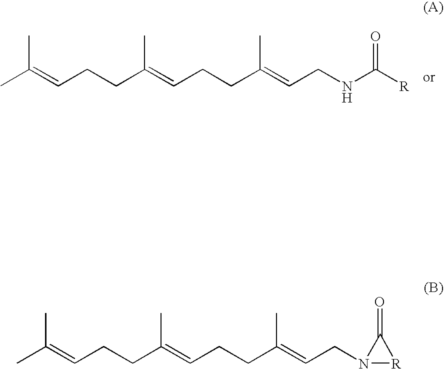
wherein R is one or more aliphatic or aromatic functional groups or substituents optionally substituted with any substituent, or a salt or analog of a compound of formula A and/or formula B.

34-42. (canceled)

43. A method for synthesizing N-farnesylphthalimide (compound I), comprising activating farnesyl alcohol with DIAD/Ph₃P and treating the product with phthalimide; or
for synthesizing N-methyl-N′-farnesylphthalamide (compound II) and/or N,N′-bisfarnesylphthalamide (compound III), comprising treating N-bisfarnesylphthalamide (compound I) with methyl amine; or
for synthesizing farnesylamine acetate (compound IV), farnesylamine propionate (compound V), and/or farnesylamine palmitate (compound VI), comprising acylation of franesyl amine with acid anhydride.

44-45. (canceled)

46. A method for inhibiting the proliferation of a cell, wherein the method comprises contacting the cell with an effective amount of at least one compound of formula A and/or formula B, or a composition comprising a compound of formula A and/or formula B and a pharmaceutically acceptable carrier: 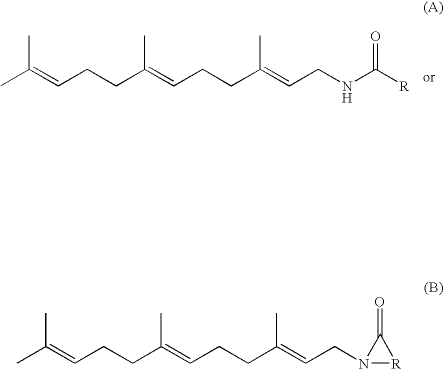
wherein R is one or more aliphatic or aromatic functional groups or substituents optionally substituted with any substituent, or a pharmaceutically acceptable salt or analog of a compound of formula A and/or formula B.

47-53. (canceled)

54. The method according to claim 46, wherein the cell is a cancer cell or a fungal cell.

55-56. (canceled)
